(19) United States
(12) Patent Application Publication
(10) Pub. No.: US 2003/0032601 A1
(43) Pub. Date: Feb. 13, 2003

(54) Method for isolating sponge collagen and producing nanoparticulate collagen, and the use thereof

(76) Inventors: Jorg Kreuter, Homburg (DE); Werner Muller, Wieshaden (DE); Dieter Swatschek, Mainz (DE); Wolfgang Schatton, Eschborn (DE); Maria Schatton, Eschborn (DE)

Correspondence Address: Norris McLaughlin & Marcus, 30th Floor, 220 East 42nd Street, New York, NY, 10017, US

(21) Appl. No.: 10/204,873

(22) PCT Filed: Feb. 28, 2001
(86) PCT No.: PCT/EP01/02228

(30) Foreign Application Priority Data
Mar. 3, 2000 (DE) 100 10 113.5

(51) Int. Cl. A61K 38/39

(52) U.S. Cl. 514/21; 530/356

(57) ABSTRACT

The invention relates to a method for the simplified isolation in high yields of sponge collagen, especially from marine sponges, and to the production of collagen nanoparticles from collagen. The invention further relates to the use thereof for influencing cell-dependent processes in vitro and in vivo, especially when orally or topically administered to treat inflammatory, preferably cyclooxygenase-dependent diseases.

The present invention relates to the method described in the claims for a simplified and thus commercially usable isolation of sponge collagen, in particular from marine sponges, and especially sponges from the category of the Chondrosiidae, as well as for the production of collagen nanoparticles from collagen.

The invention furthermore relates to the use of such collagen for the production of a substance for influencing cell-dependent processes in vitro and in vivo. In particular the application relates to the use of such isolated collagen and/or of collagen nano- or microparticles for the preparation of creams, ointments, suspensions, tablets, capsules, also the delayed release, implants, band aids, foams etc. for covering wounds, active ingredient carriers in parenterals and enterals, eye drops, nano-capsules as active ingredient carriers and carriers for active ingredients through the skin and mucous membranes as well as vessels and organ membranes. The collagen produced pursuant to the invention is suited for the production of substances for external and internal, preferably oral and topical applications for the treatment of inflammatory, especially also cyclooxygenase-dependent diseases in vivo as well as furthermore for promoting the growth of permanent and neuronal cells in vitro.

Collagen is a bio-degradable and compatible protein and is used as the starting material for a broad range of applications in the pharmaceutical industry, in cosmetics and food chemistry.

So far collagen has been gained from animal skin and the bones of pigs, calves and cattle. Here the possibility of infecting the user with pathogenic germs, viruses and above all with BSE (bovine spongiforme encephalopathy) represents a decisive disadvantage.

Sponges are more than 600 million years old and thus represent the oldest members from the group of the metazoans. Like all multiple-cell animals, sponges contain the connective tissue protein collagen. Sponge collagen represents a water-insoluble protein, which has the typical amino acid composition of familiar collagen types. Glycine, for example, represents about every third amino acid, and the percentages of proline as well as hydroxy-proline are high.

Sponge collagen can be isolated with various methods. So far fresh material has been used for this with little practical relevance. The cleaning method for such isolated sponge collagen has been largely relatively complex from an industrial point of view (e.g. gel filtration). And the yield as well was not yet satisfactory. For example, from 1000 g sponge material, 21 g sponge collagen was isolated.

This is described by B. Diehl Seufert et al. in J. Cell Sc. 79, 271-85 (1985). The isolation of collagen from the Geodia cydonium and Chondrosia renifornis sponges with natural amino acid composition while avoiding denaturation reagents takes place through the suspension of homogenized fresh material in Tris-HCl buffer, pH adjustment to 9, centrifugation and homogenization. Cleaning occurs with gel filtration, wherein as mentioned above, 1.7 or 3.1% collagen is obtained.

There is a need therefore for a simple and economical method for isolating collagen from sponges, while simultaneously avoiding the disadvantages for collagen gained from animal starting material, and obtaining high yields.

Beyond that, it is particularly advantageous to produce the smallest particles of collagen possible.

The production of collagen micro-particles has been described so far for native calf collagen. However it was only possible to produce micro-particles in the range from 3 to 40 μm, e.g. as described in B. Rossler, J. Kreuter and D. Scherer ‘Collagen Microparticles, Preparation and Properties’, J. Microencapsulation, Vol. 12, No. 1, 49-57 (1995). This range however is not very suitable for pharmaceutical and cosmetics applications. The intravenous application is associated with the risk of embolism. The application on skin or on the eye slows down the release of attached substances so that it does not occur within a useful time frame before the preparation is washed off or is removed e.g. through drainage from the eye.

Additionally, the relatively large particle size can create an unpleasant scratchy sensation. For this reason it is necessary to restrict the range, i.e. keep it below 3 μm or smaller.

It is therefore the object of the present application to develop a product-oriented isolation method for sponge collagen, which leads to good yields of a product with a high collagen percentage in a simple and effective manner, wherein minimal toxicological risk should exist. At the same time, the starting material should be inexpensive and practice-relevant so that the addition of deep-frozen fresh material as starting material can be foregone (as it has been used until now).

Furthermore collagen particles are to be produced which have as small a size as possible, particularly which are in the micro and nano range.

This task is resolved with the invention in that the fresh starting sponge material, which does not have to be frozen, is placed in alcohol, subsequently washed with water, treated with an extractant, preferably with a pH value of 7-12, in particular 8-10, above all 9-95 or 9.5 and the resulting collagen extract is worked up. This takes place preferably by increasing the pH value of the suspension to a pH of 8-11, in particular 9-10, above all 9, stirring, centrifugation and subsequently through a reduction of the pH value of the residue, centrifugation and isolation of the precipitate.

This process eliminates another cleaning process, such as e.g. through gel filtration or the like. The cleaned collagen product, which can be freeze-dried for conservation purpose if desired, is then obtained in a high yield.

Suitable starting materials are the conventional, familiar, especially marine sponges such as, for example, Demospongiae [Rupert Riedel, Fauna und Flora des Mittelmeeres (Fauna and Flora of the Mediterranean Sea), Paul Parey Publishing Company, 1983], particularly such with high collagen percentage such as sponges of the category of the Geodiidae, Dysideidae, Spongiidae, Suberitidae, Oscarellidae, Axinellidae and others, above all from the group of the Chondrosiidae, which beyond that have minimal toxicological risk. Chondrosiidae or also Axinellidae are particularly preferred.

The familiar straight-chain or branched-chain aliphatic C₁-C₁₅ alcohols such as ethanol, isopropanol, butanol, tert. butanol, pentanol, cyclohexanol, hexanol etc are suitable alcohols. Beyond that additives of aromatic alcohols such as thymol as well as benzyl alcohol and combinations thereof, e.g. among each other or with the aliphatic alcohols, in particular ethanol or isopropanol, are also possible.

C₁-C₄ aliphatic alcohols, and especially ethanol, are particularly preferred. The quantity of alcohol depends on the quantity of starting material since said material is placed in alcohol.

Suitable extractants are e.g. familiar systems such as Tris-HCl buffer (preferably containing 10 mM EDTA, 8M-urea, 100 mM-2-mercaptoethanol) or alkali-containing buffer systems, e.g. potassium carbonate-containing, phosphate-containing, nitrogen- or sulfate-containing buffers with a suitable pH value. In this case, the substance is used in excess in relation to the weight of the starting material that is used, especially at 2-8 times the weight quantity, especially 3-6 times and above all 5 times the quantity.

Such buffers are generally known and described for example in the European Pharmacopeia.

The pH value can be adjusted or reduced with known means. Suitable for this are e.g. buffer solutions such as the above-mentioned nitrogen, phosphate, sulfate, potassium carbonate-containing or organic buffers such as acetate or citrate containing buffer systems with suitable pH value.

Precipitation of the collagen from the residue after centrifugation can be accomplished through a pH value reduction, especially to values between 2 and 6, especially 5-3 and above all pH 4, through appropriate means such as organic acids, e.g. acetic acid (e.g. 100%), citric acid, ascorbic acid, propanoic acid, formic acid or lactic acid etc. or through diluted inorganic acids, e.g. hydrochloric acid, phosphoric acid, sulfuric acid and the like. The subsequent isolation of the precipitate can occur with renewed centrifugation, wherein preferably—as is also the case with the initial centrifugation—the temperature can be lowered, especially to values smaller than 10° C.-1° C., preferably 6° C.-1° C., especially 2° C.

The product that is obtained (collagen sediment) is pure and can be freeze-dried for conservation if desired.

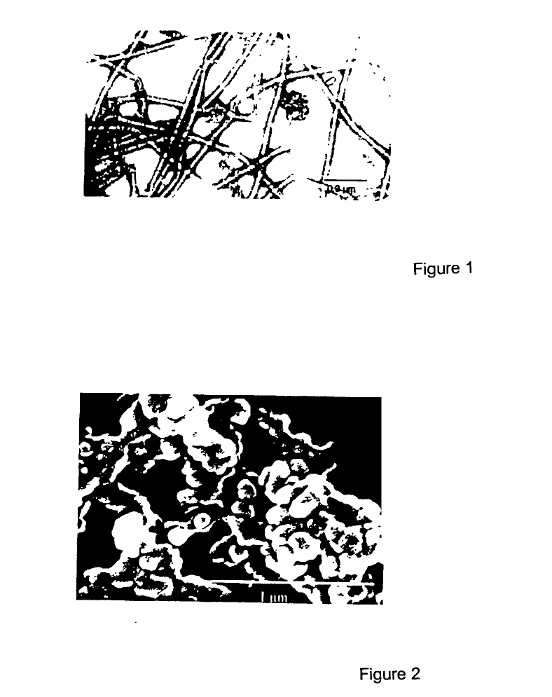

FIG. 1 (see example 2) shows such a lyophilized product obtained pursuant to the method of the invention, wherein the periodic cross stripes of the collagen filtration that is typical for collagen can be recognized.

With the invented method it is thus possible to obtain in high yields pure collagen without complex cleaning steps.

Surprisingly, it has been shown that the typical features for collagen, such as periodic cross stripes of the fibrils, remains present and thus is natural.

The method is particularly suited for industrial ‘scaling-up’. The cleaning of isolated sponge collagen could be simplified according to the invention in favor of a practice-oriented method. The yield of sponge collagen is very high at about 10-40%, or 30% on average. Since the sponge species utilized are safe from a toxicological point of view, especially e.g. when used for edible sponges from the families of the Chondrosiidae or Axinellidae, one can assume that the sponge collagen obtained this way is very compatible and largely safe for humans from a toxicological point of view. Beyond that, the isolated sponge collagen exhibits considerable advantages over pig, calf and cattle collagen with regard to the BSE problem.

As mentioned above it is beneficial to produce collagen material in particularly small particle sizes. So far native calf collagen has been emulsified and cross-linked with glutardialdehyde solution. The particles obtained had a size of 3-40 μm.

According to the invention now particle sizes of less than 3 μm can be achieved for the first time.

Collagen, obtained based on familiar methods from starting materials of various, e.g. animal origins as described above from calves, pigs, cattle or sponges, especially such obtained from sponges with the invented method, is dispersed e.g. in water and initially homogenized. The pH value can be adjusted preferably then or if necessary before the first homogenization to pH 7-11, especially 8-10, preferably 9-10, above all 9.5, and the substance can be homogenized again. Subsequently it is emulsified, through the addition of an emulsifying agent and a fatty phase such as e.g. through the addition of paraffin and Span ® or e.g. through other W/O emulsifying agents such as cetylstearyl alcohol, glycerin monostearate or Span ® 85 (Sorbitantrioleate), or similar products such as Arlacel 85, Span 65 (Sorbitantristearate), Arlacel 65, propylene glycol monostearate, sorbitan monooleate, Span 40, Arlacel 40, Span 20 or possible O/W emulsifying agents of the familiar kind (polyethylene glycols with suitable ethoxylation degree; such as e.g. with HLB value 8-13, e.g. polyoxyethylene glycol-400-monostearate, oleyl ether, monolaurate, sorbitan monolaureate, sorbitan tristearate) in a weight of more than 2 to 5 times the quantity of the collagen dispersion. Apart from paraffin, similar liquid hydrocarbons, such as e.g. isoparaffin, dioctylcyclohexane (Cetiiol® S), isohexadecane (Arlamol® HD), triglycerides such as Miglyol®, squalane or squalene or mixtures thereof are suited. The quantity of the fatty phase is specified together with the quantity of emulsifying agent and is, as mentioned above, 2-5 times the collagen quantity.

Subsequently the product is cross-linked with an excess, i.e. more than twice the amount than before, i.e. at least 2 to 6 times the weight, preferably 3-5 times, especially 4 times, in relation to the collagen quantity that is used, of cross-linking agents such as e.g. bi-functional acid chlorides, aldehydes, especially glutardialdehyde (e.g. a 25% aqueous solution thereof) or maleic acid dialdehyde or dichloride.

The reaction is worked up in a suitable fashion, initially interrupted e.g. through the addition of H₂O₂. For this, a considerably higher weight is used than previously, i.e. 2-6 times, preferably 3-5 times, especially 4 times the quantity in relation to the collagen quantity that is used. Alternatively, other familiar suitable oxidation agents such as HNO₃ can also be used to terminate the process. Reconditioning occurs e.g. through centrifugation, suspension of the sediment in alcohol, e.g. isopropanol, renewed centrifugation and additional runs through this cycle. In the last cycle, suspension with a weak acid occurs, e.g. ascorbic acid or another organic acid, e.g. citric acid, lactic acid, formic acid and the like. This sediment is then heated, e.g. to temperatures >50° C., especially to 75° C., stirred again for several hours (e.g. 24), centrifuged and rinsed with water.

The material that is obtained can be freeze-dried. FIG. 3 depicts the size distribution of nano-particles, which were produced as described in the subsequent example 3. This shows that more than 95% of all particles are nano-particles.

Surprisingly the smallest particulate collagen can be produced with the above-described method when the starting material, which is suitably dispersed e.g. in distilled water, is initially homogenized before emulsification and cross-linkage. Homogenization suitably occurs through ultrasound treatment, Ultraturrax or high pressure homogenizer or micro-fluidizer. After adjusting the pH value e.g. with potassium carbonate from pH 4 to pH 8, preferably another homogenization process can be conducted. Cross-linkage occurs with a larger quantity of cross-linking agents than has been used until now.

Collagen is particularly preferred as the starting material, obtained pursuant to the above-described isolation method of the invention from marine sponges of the family of the Chondrosiidae.

This new method for producing collagen micro-particles from collagen, especially from sponge collagen such as e.g. from marine sponges of the family of the Chondrosiidae, consequently makes the production of particles in a size range from 150 nm to 3 μm possible, in contrast to familiar methods for producing collagen, which enable micro-particles of only 3-40 μm.

The collagen and nano-particulate collagen obtained in the manner of the invention find broad applications in pharmacy, medicine, cosmetics and food chemistry such as creams, ointments, for applications on skin and mucous membranes, suspensions, tablets, capsules, also with delayed release, implants, band aids, foams, sponges, fleece and the like for covering wounds, active ingredient carrier in parenterals and enterals, eye drops, nano-capsules as active ingredient carrier and carrier for active ingredients through the skin and mucous membranes as well as vessels and organ membranes. These substances were prepared in the familiar fashion. The production methods for this are e.g. described in DAB and are known both for the manufacture of ointments, creams as well as of tablets, etc. wherein conventional quantities are used.

Surprisingly it has been shown that the collagen prepared pursuant to the invention, especially the nano-particulate collagen, exhibits an anti-inflammatory effect especially in the case of topical and also oral application apart from intravenous or intraperitoneal application. It turns out that the substance is an inhibitor of cyclooxygenase and also has an anti-oxidative effect. Particularly surprising is the anti-inflammatory effect e.g. for joint problems, arthritis and especially analogous diseases of the locomotor system, in particular when the substance is administered orally. In this case, the above-described, especially marine sponge collagen is suspended as such or in the freeze-dried state in a suitable beverage, such as water or juices, and administered as a beverage, e.g. in quantities of 2-10 g, especially 2-6 and preferably 3-5 g/day. It can be available as a solid as well as a powder or granulated collagen as described below, preferably in a quantity of 1-10, especially 2-8 and above all 3-5 g/250 ml of fluid, preferably water.

As a substance which can be topically applied, both a penetration reinforcement of another active substance and/or in combination therewith, such as, for example, of a vitamin (vitamin A, C, E or their mixtures) or other topically active substances for the treatment of the skin such as avarol, avarone or plant extracts, such as Extr. Cepae or Extr. Echinaceae pallidae, as well as in particular an anti-inflammatory effect can be observed. The described collagen is especially suited for the treatment of skin changes, e.g. of degenerative type, or when the skin is damaged due to outer or inner influences, e.g. for the treatment of erythema after sunburns, UV radiation or injuries or psoriasis. The substance is also suitable for the indications of this type mentioned below. The topical agent can be available in the form of creams, ointments, lotions on a familiar basis, or especially in the form of a gel such as a hydrogel e.g. on the basis of polyacrylate or an oleogel e.g. made of water and Eucerin.

The employed oleogels made of an aqueous and a fatty phase are based particularly on Eucerinum anhydricum, a basis of wool wax alcohols and paraffin, wherein the percentage of water and the basis can vary. Furthermore additional lipophilic components for influencing the consistency can be added, e.g. glycerin, polyethylene glycols of different chain length, e.g. PEG400, plant oils such as almond oil, liquid paraffin, neutral oil and the like. Such prescriptions are generally known and described in DAB10, or in the European Pharmacopeia, current edition, e.g. 2000. The hydrogels can be produced through the use of gel-forming agents and water, wherein the first are selected especially from natural products such as cellulose derivatives, such as cellulose ester and ether, e.g. hydroxyethyl-hydroxypropyl derivatives, e.g. tylose, or also from synthetic products such as polyacrylic acid derivatives, such as Carbopol or Carbomer, e.g. P934, P940, P941. They can be produced or polymerized based on known regulations, which are described in current pharmacopoeias such as e.g. DAB10 or the European Pharmacopeia, current edition, e.g. 2000, from alcoholic suspensions by adding bases for gel formation.

Per 100 g of gel, the gels comprise 0.01-30 g, especially 0.01-10 g and above all 0.01-8 g and preferably 0.1-5 g collagen, i.e. accordingly 0.01-30; 0.01-10; 0.01-8; 0.1-5%.

This way a substance can be made available, which does not exhibit the side effects of existing substances used for the treatment of inflammatory diseases, such as e.g. NSDAIs, and are above all orally well tolerated.

The above-described agents are therefore suited for compositions for the treatment of inflammations in a living being and for the treatment of other inflammation-related malfunctions. Examples for inflammations and inflammation-similar malfunctions are arthritis, including rheumatoid arthritis, spinal joint problems, gout, systemic lupus erythematosis, osteoarthritis and juvenile arthritis, furthermore asthma, bronchitis, menstrual cramps, tendonitis, bursitis and conditions associated with the skin such as psoriasis, excema, burns and dermatitis. The described agents are also suitable for the treatment of gastrointestinal conditions, such as inflammatory intestinal disorder, Crohn's disease, gastritis, irritable bowel syndrome and ulcerative colitis, for the treatment of inflammation in diseases, such as vascular illnesses, periarteritis, Hodgkin's disease, sclerodema, rheumatoid fever, type I diabetes, myasthenia gravis, sarcoidosis, nephrotic syndrome, Behcet's syndrome, polymyositis, bleeding gums, hypersensitivity, conjunctivitis, swelling occurring after injuries, myocardialischemia etc.

Additionally this invention comprises a category of compositions, containing a collagen produced as described, especially marine sponge collagen, in particular nano-particulate collagen, together with one or more non-toxic pharmaceutically tolerable vehicles and/or diluting agents and/or adjuvants (described in the following under the collective term “vehicle” materials) and possibly other active ingredients. The substances pursuant to the invention can be administered as mentioned above in any suitable way and at a dosage that is effective for the intended treatment. The substance can, for example, be administered intravascularly, intraperitoneally, subcutaneously, intramuscularly, especially orally or topically.

For oral administration, the agent can assume the form of e.g. a tablet, capsule or suspension. In this case the substance is preferably produced in the form of one dosage unit. The substance can additionally be administered through injection as a composition, wherein for example saline solution, dextrose or water can be used as a suitable vehicle.

The quantity of the administered substance and the dosage schedule for the treatment of an illness with the described substance depend on a multitude of factors, including the age, weight, sex and medical condition of the patient, the severity of the illness, the administration route and the frequency of administration, as well as on the compound in question used, and can therefore fluctuate greatly. The substances of the invention can generally be combined with one or more adjuvants, which are suitable for the specified administration route. If administration occurs per os, the described collagen product can also be mixed with lactose, saccharose, starch powder, cellulose ester or alkane acids, cellulose alkyl ester, talcum, stearic acid, magnesium stearate, magnesium oxide, sodium and calcium salts of phosphoric and sulfonic acids, gelatins, gum arabic, sodium alginate, glycols, polyvinyl pyrrolidone and/or polyvinyl alcohol and then be put in tablets or capsules for easy administration. Such capsules or tablets can contain a controlled release formulation, as can be provided in a dispersion of the substance in hydroxypropyl methyl cellulose. Formulations for parenteral administration can exist in the form of aqueous or non-aqueous isotonic sterile injection solutions or suspensions. These solutions and suspensions can be produced from sterile powders or granules with one or more vehicles or diluents, as they were mentioned for the use in formulations for oral applications. The agents can be dissolved in water, polyethylene glycol, propylene glycol, ethanol, corn oil, cotton seed oil, peanut oil, sesame oil, benzyl alcohol, sodium chloride and/or various buffers. Other adjuvants and types of administrations are known.

The above-described collagen is used in a cell suspension in a quantity of 0.01-150 μg/ml, especially 0.1-100 μg/ml and above all 1-60 μg/ml of cell suspension as a means for influencing cell-dependent processes in vitro. Permanent cells such as CEM cells or neuronal cells like cortical cells are particularly suited as cells to promote growth. Suitable collagen is either the directly isolated product as described above or the nano-particulate product of the invention in accordance with the present application. The collagen is then added either in a solid state to the cell sample and is available as suspension, or is dissolved in an alkaline aqueous medium (pH value greater than 7, especially greater than 8), placed into the sample container, acidified until the collagen precipitates, and then the cell sample is added.

The invention is explained in more detail based on the following examples.

### EXAMPLE 1

### Method for Isolating Sponge Collagen from Marine Sponge of the Family of Chondrosiidae

The sponge material is collected, broken down into coarse pieces and immediately placed in ethanol for conservation. The sponge pieces are washed three times with distilled water and homogenized with a mixer. The broken down material is mixed with five times the weight of an extractant (pH 9-5; 10 mM-EDTA, 8 M-urea, 100 mM-2-mercaptoethanol). The pH value of the resulting dark brown suspension is adjusted with tris from pH 7 to pH 9. This suspension is stirred for 24 hrs at room temperature and subsequently centrifuged (5000 g, 6 min., 2° C.). The pellet is discarded. The pH value of the residue is adjusted to pH 4 with 100% acetic acid. A precipitate forms, which is collected (20,000 g, 20 min., 2° C.). The pellet is mixed with water and centrifuged again (20,000 g, 10 min., 2° C.). This results in a clear, slightly colored residue as well as the cleaned collagen sediment, which can be lyophilized for conservation purposes.

The collagen yield is around 30% (freeze-dried collagen sediment in relation to freeze-dried sponge material).

### EXAMPLE 2

### Electron-microscopic Characterization of the Isolated Sponge Collagen (TEM/Transmission Electron Microscopy)

For examination purposes, the freeze-dried collagen sample is negatively contrasted with a 2% phosphotungstic acid based on the so-called single droplet method (see J. R. Harris, Negative staining and cryoelectron microscopy. The thin film techniques, Royal Microscopial Society Microscopy Handbook No. 35. BIOS Scientific Publishers Ltd., Oxford, UK).

In the enlargement, one will recognize the periodic cross stripes of the collagen fibrils, which are typical for collagen, in the TEM image in accordance with FIG. 1.

### EXAMPLE 3

### Preparation of Collagen Micro-particles from Sponge Collagen of the Family of Chondrosiidae

Initially a 0.75% sponge collagen dispersion is produced by mixing the freeze-dried material with distilled water and homogenizing it with an Ultra-Turrax. The pH value of the dispersion is adjusted to pH 9.5 with potassium carbonate. Subsequently the dispersion is homogenized a second time with the Ultraturrax.

A mixture is prepared from 10 g Span® and 250 g liquid paraffin, to which 85 ml of the 0.75% collagen dispersion is added. This mixture is emulsified for 10 minutes at about 6000 rpm with an Ultraturrax (Ika Factory, Staufen, Germany). Subsequently, the emulsion is stirred constantly with wing stirrer. For the purpose of cross-linkage of the collagen chains, 12 g of a 25% aqueous glutardialdehyde solution is added. This reaction is completed after 12 minutes through the addition of 16 g of a 30% aqueous hydrogen peroxide solution. The preparation is stirred for an additional 15 minutes. The emulsion is diluted with 100 ml 2-propanol.

For cleaning the CMPs, the emulsion is centrifuged (30 minutes/10444 g), the oily residue is discarded and the sediment is suspended in 50% 2-propanol. Subsequently the suspension is centrifuged again. This cleaning step is repeated. The CMP sediment that is obtained is suspended in a 4% aqueous ascorbic acid solution. This suspension is heated (75° C., 30 minutes) so as to destroy residual quantities of oxidizing substances. The mixture is stirred for 24 hrs on a magnetic stirrer at room temperature. The CMP is subsequently cleaned through centrifugation (30 minutes, 10444 g) and then washed twice with water.

The material is freeze-dried. The CMP yield is around 10%.

### EXAMPLE 4

### Scanning Electron Microscopic Examination of the Collagen Micro-particles Pursuant to Example 3

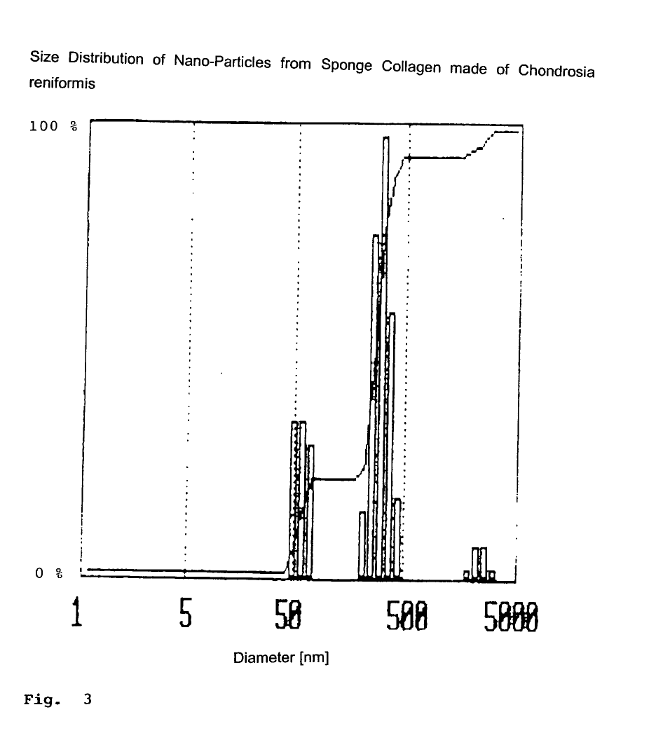

The collagen micro-particles were coated with about a 4 nm thick platinum film and examined with the SE 4500 Hitachi S scanning electron microscope. The size of the spherical collagen particles lies at 120 to 300 nm (FIG. 2).

### EXAMPLE 5

### Particle Size Determination/Photon Correlation Spectroscopy (PCS) of Collagen Particles Pursuant to Example 3

The particle size determination process was conducted with the help of photon correlation spectroscopy (PCS). The lyophilized sponge collagen was resuspended in filtered water (pH 9.8, adjusted with K₂CO₃) before measurement. This resulted in a particle size distribution of 150 nm to 3 μm. While the lower limit agrees quite well with the results of the scanning electron microscopy, at 3 μm the upper range is clearly above the value of 300 nm. This could be due to agglomerates, which form in water during redispersion and cannot be dissolved even in the ultrasonic bath.

FIG. 3 shows the size distribution of the nano-particles produced pursuant to example 3 and shown in FIG. 2. As can be seen, more than 95% of all particles are nano-particles. 25% of all particles have an average diameter of 50 nm and are more than 70% in the range of 300 nm (abscissa=diameter of particles [nm], ordinate=parts in %).

### EXAMPLE 6

### Thio-barbituric Acid (TBA) Antioxidant Test with Collagen from Chondrosia reniformis

The test is based on the formation of a reaction product, measurable at 532 nm, of thio-barbituric acid (TBA) with malondialdehyde (MDA), which arises among other things in the oxidative decomposition of multiple unsaturated fatty acids, as they are contained in trilinolenin (Sigma).

Trilinolenin and the test substances, sponge collagen produced from Chondrosia reniformis as described in example 1, and butylhydroxytoluene [BHT] are dissolved in DMSO.

400 μl of trilinolenin are incubated in 2 ml HAM F10—Medium for 4 hours at 37° C. in the presence of DMSO as control, or DMSO-containing substance solutions.

For determining the overall TBA-reactive material, 1.0 ml of the incubation batch is removed and mixed with 1.5 ml of TBA solution (0.67% in 0.05 M NaOH) as well as with 1.5 ml of tri-chloracetic acid (20%).

After a 60-minute incubation in a boiling water bath the samples are cooled down and centrifuged for 10 minutes at 2000 rpm. The visual density is determined at 532 nm. Fresh tetramethoxypropane, which is obtained under the test conditions from MDA, serves as calibration standard. The results are calculated as μmol MDA equivalent. Triple determination processes are conducted.

Table 1: effect of sponge collagen in thio-barbituric acid (TBA)—antioxidant test (% inhibition (±SD) of the formation of TBA reactive material compared to BHT as standard substance)

TABLE 1

Concentration [μM]

| Substance | 5 | 10 | 20 | 50 |
|---|---|---|---|---|
| BHT | 71 (±1.5) | 73 (±2.1) | 79 (±0.3) | 85 (±0.5) |
| Collagen* | 18 (±1.3) | 37 (±2.3) | 41 (±3.9) | 47 (±1.8) |

*collagen = collagen from Chondrosia reniformis

Surprisingly, collagen from Chondrosia reniformis inhibits the formation of TBA-reactive material. This was not known until now. The potency is about 50% of the standard BHT substance. Collagen from Chondrosia reniformis shows medium effects with a flat dosage effect relation curve in the concentration range that was examined.

### EXAMPLE 7

### In vitro Inhibition of Cyclooxygenase

The cyclooxygenase activity was determined in monocytes in the presence or absence of the agonist lipopolysaccharide [LPS] (L-4130; Sigma, St. Louis). For this purpose, human monocytes were kept in a density of 106 cells per ml in RPMI-1640 medium for 24 hrs. Subsequently 30 μM arachidonic acid was added to the cultures and the enzyme activity was measured 30 minutes later based on the production rate of prostaglandin E2.

Table 2:

Effect of sponge collagen, produced as described in example 1, on the cyclooxygenase activity in human monocytes.

The values represent mean values (±S.D.) of five independent results.

TABLE 2

| Addition | Collagen | Cyclooxygenase Activity |
|---|---|---|
| of LPS | (μg/ml) | (pg PGE2/106 cells) |
| none | 0 | 88 ± 10 |
| | 1 | 71 ± 9 |
| | 5 | 52 ± 6 |
| | 0 | 243 ± 22 |
| | 0.5 | 187 ± 19 |
| | 1.0 | 79 ± 7 |
| | 5.0 | 61 ± 7 |

Result:

After the addition of LPS, an increase in the cyclooxygenase activity occurs from 88 to 243 pg PGE 2 per 106 cells within 30 minutes. Increasing concentrations of sponge collagen from Chondrosia reniformis eliminate the stimulating effect of LPS on the cyclooxygenase. At a concentration of 3 μg/ml, the induction of the cyclooxygenase is reduced to 61%.

### EXAMPLE 8

### Sponge Collagen for Chronic-inflammatory and Degenerative Motion Problems

The Chondrosia reniformis sponge is being eaten still today. 10 patients with chronic arthritis therefore agreed to an oral treatment. 5 g collagen from Chondrosia reniformis, produced as described in example 1, is to be taken suspended in a beverage three times a day for a period of 4 weeks. All patients unanimously reported, after a short period of time, decreased pain in the joints and improved motion.

### EXAMPLE 9

### Penetration of the Skin of Naked Mice in vitro

Nano-particles gained from the Chondrosia reniformis collagen, produced as described in example 3, were loaded with 14C-marked retinol (charge rate 15%) and worked into an oleogel made of water/Eucerin as described above (collagen: 1%, added as 10% suspension).

Diffusion cells according to Franz [n=6] were filled with physiological saline solution and the acceptor cells were covered with a freshly prepared section of the skin of naked mice. 100 mg of the gel loaded with 14C-marked retinal was applied onto the skin sections. Appropriate gels with 14C-marked dissolved retinal served as controls [n=6].

After 2, 4, 8, 16, 24 and 36 hours the 14C activity in the acceptor cell was determined.

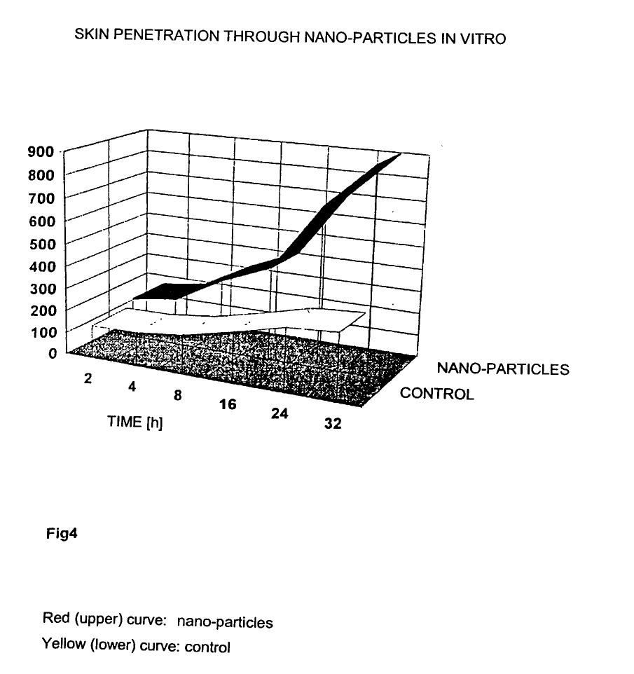

The results are shown in FIG. 4 and clearly prove the superiority of sponge collagen nano-particles in the penetration of mice skin compared to a hydrogel, which contains the marked substance only in dissolved form.

### EXAMPLE 10

### Examination on UV or Heat-induced Erythema of Human Skin

The examinations were conducted on volunteers, who suffered acutely from sunburns [n=7] or burns [n=3]. Sponge collagen nano-particles [1%] in accordance with example 3 were topically applied in a polyacrylate gel, produced as described above (1% collagen, added as 10% suspension).

The clinical effect of the applied gel on the UV or burn-related change in skin was evaluated macroscopically 1 hour later after a large scale application of the gel as well as 2, 4, 6 and 24 hours later, and the patient was questioned about his/her condition.

Result:

Sponge collagen reduced the erythema quickly during the first few hours after the erythema-triggering event. The one-time application had a long-term inhibiting effect of up to 5 hours. This could be reinforced by incorporated familiar topical inflammation-inhibiting substances into the nano-particles, such as vitamin E, Avarol, Avarone or plant extracts, such as extracted Cepae or extracted Echinaceae pallidae.

### EXAMPLE 11

### Toxicity Examinations on the Collagen Pursuant to the Invention

In order to determine toxicity, the influence of sponge collagen onto cellular growth in cultures was examined on a permanent cell line and on freshly prepared neuronal cells.

1. CEM Cells (Permanent Human Leukemia Cells)

CEM cells [Sechoy et al., Exp. Cell Res. 185: 122, 1989 and Avramis et al., AIDS 3: 417, 1989] were either not treated with sponge collagen (control sample) or incubated with various concentrations thereof.

Cell growth was the examination parameter. CEM cells were used at a concentration of 0.2×10⁶ cells/ml culture medium inoculation of the culture. After a 4-day incubation, the density of the CEM cells was 1.9×10⁶ cells/ml. This value forms the control value. After an additional 4 days the cell density was determined again.

2. Neuronal Cells

Cortical cells (neurons) were prepared from the brains of newborn Wistar rats and kept under cell culture conditions. The culture medium used was MEM medium (with 10% horse serum), and it was incubated at 90% humidity and 10% CO₂ atmosphere. Generally after 48 hours of incubation in culture, the cells were used for the experiments. The culture contain neurons in an overwhelming concentration (more than 70%) and about 20% GFAP positive astrocytes (Müller et al.: Europ. J. Pharmacol.—Molec. Pharmacol. Sect. 226: 209-214,1992). After an additional 4 days, the cell density was measured experimentally. The results are shown in table 3.

TABLE 3

| | Concentration of the | |
|---|---|---|
| | Sponge Collagen | Cell Concentration × |
| | (μg/ml) | 1,000,000/ml |
| Control | 0 | 1.9 ± 0.24 |
| 1. CEM Cells | 0.1 | 2.0 ± 0.2 |
| | 1.0 | 2.5 ± 0.3 |
| | 10.0 | 2.9 ± 0.3 |
| | 100.0 | 2.7 ± 0.2 |
| 2. Neuronal Cells | 0.1 | 2.3 ± 0.2 |
| | 1.0 | 2.7 ± 0.3 |
| | 10.0 | 3.4 ± 0.2 |
| | 100.0 | 2.5 ± 0.2 |

The results of the table represent mean values of 6 parallel experiments with standard deviations. The significance was determined with the Student t-test (Sachs, L.: Angewandte Statistik [Applied Statistics], Berlin: Springer-Verlag, pp. 209-216; 1984).

It is obvious that sponge collagen has no negative effects on the growth rate of permanent cells and neuronal cells in a primary cell culture. On the other hand, in the presence of sponge collagen, CEM cells and neuronal cells increase their growth rates significantly above their control values (p<0.001). It is only at concentrations around 100 μg/ml that no additional growth increase takes place.

This clearly shows that the collagen of the invention on one hand has an inhibiting effect on monocyte-dependent cells and enzymes deducible thereof such as cyclooxygenase for example, without being toxic, and on the other hand has a growth-enhancing effect on permanent cells and neuronal cells. Thus the collagen that is produced pursuant to the invention can be used not only for the in vivo treatment especially of cyclooxygenase-dependent illnesses, but above all also preferably for the in vitro influencing of permanent or neuronal cells. In these cases it can be employed in the above-mentioned quantities, especially between 0.01, preferably 0.1, and 100 μg/ml cell suspension.

1.) Method for isolating sponge collagen, characterized in that the starting material is placed in alcohol, subsequently washed, treated with an extractant, and that the collagen extract obtained this way is then reprocessed.

2.) Method pursuant to claim 1, characterized in that sponge collagen from marine sponges is isolated.

3.) Method pursuant to claim 1, characterized in that sponge collagen from Demospongiae, preferably Chondrosiidae, is isolated.

4.) Method pursuant to one of the claims 1-3, characterized in that ethanol is used as the alcohol.

5.) Method pursuant to one of the claims 1-4, characterized in that base buffer systems, preferably Tris buffers, are used as extractant.

6.) Method pursuant to one of the claims 1-5, characterized in that the extractant is used in excess amounts in relation to the weight of sponge starting material.

7.) Method pursuant to one of the claims 1-6, characterized in that, for the purpose of reprocessing, the pH value of the collagen extract is increased, the suspension is stirred, centrifuged, the residue is acidified and the precipitate is isolated.

8.) Method pursuant to one of the claims 1-7, characterized in that the isolated collagen product is freeze-dried.

9.) Usage of a collagen product, manufactured pursuant to the method in accordance with one of the claims 1-8, for producing a cosmetic, medical or pharmaceutical substance for topical, intravenous, intramuscular or oral applications.

10.) Method for producing nano-particulate collagen, characterized in that the starting collagen material is dispersed and homogenized, subsequently emulsified and cross-linked with an excess amount of cross-linking agent, and subsequently reprocessed.

11.) Method pursuant to claim 10, characterized in that a collagen product, as obtained pursuant to the method in accordance with one of the claims 1-8, is used as the starting material.

12.) Method pursuant to claim 11, characterized in that collagen obtained from sponges of the category of Chondrosiidae pursuant to the method in accordance with one of the claims 1-8 is used.

13.) Method pursuant to one of the claims 10-12, characterized in that collagen with a particle size of 150 nm to 3 μm is produced.

14.) Method pursuant to one of the claims 10-13, characterized in that glutardialdehyde is used as the cross-linking agent.

15.) Usage of a collagen product, produced pursuant to a method in accordance with one of the claims 1-14, for the production of a substance for influencing cell-dependent processes in vitro and in vivo.

16.) Usage pursuant to claim 15 for the production of a cosmetic, medical or pharmaceutical substance for topical, intravenous, intramuscular or oral applications in in-vivo-dependent processes.

17.) Usage pursuant to claim 16 for the production of a substance for treating cyclooxygenase-dependent illnesses.

18.) Usage pursuant to claim 16 for the production of an orally administered substance for treating illnesses of the locomotor system.

19.) Usage pursuant to one of the claims 15-18 for the production of a topically administered agent in the form of an oleogel or hydrogel.

20.) Usage pursuant to claim 15 as biochemical substance for in vitro growth-enhancing application in permanent and neuronal cells.

21). Method for in vitro growth enhancement of permanent and neuronal cells, characterized in that collagen, obtained pursuant to the method in accordance with one of the claims 1-8 or 10-14, is added to the cell suspension at a quantity of 0.1-100 μg/ml suspension.

22.) Gel in the form of oleogel or a hydrogel, containing water, a gel basis as well as collagen, produced pursuant to the method in accordance with one of the claims 1-14.

23.) Collagen suspension, containing collagen, produced pursuant to the method in accordance with one of the claims 1-14 in water at a quantity of 1-10 g collagen/250 ml.

24.) Collagen, obtained pursuant to the method in accordance with one of the claims 1-8 or 10-14.
